(19) United States
(12) Patent Application Publication
(10) Pub. No.: US 2019/0194258 A1
(43) Pub. Date: Jun. 27, 2019

(54) METHOD FOR CONTROLLING IMPURITY OF CYCLOSPORIN A EYE GEL

(71) Applicant: ZHAOKE (GUANGZHOU) OPHTHALMIC DRUG COMPANY LIMITED, Guangzhou (CN)

(72) Inventors: Gang Li, Hefei (CN); Kailei Cao, Hefei (CN); Xiaoyi Li, Hong Kong (HK); Xiangrong Dai, Hefei (CN); Lei Yin, Hefei (CN); Juan Ling, Hefei (CN)

(73) Assignee: ZHAOKE (GUANGZHOU) OPHTHALMIC DRUG COMPANY LIMITED, Guangzhou (CN)

(21) Appl. No.: 16/228,802

(22) Filed: Dec. 21, 2018

(30) Foreign Application Priority Data
Dec. 21, 2017 (CN) 201711391728.1

(51) Int. Cl. C07K 7/64; C07K 1/16; B01D 15/16; B01D 15/18; B01D 15/42; B01J 20/10

(52) U.S. Cl. CPC C07K 7/645; C07K 1/16; B01D 15/163; B01J 20/291; B01D 15/18; B01D 15/426; B01J 20/103; B01D 15/161

(57) ABSTRACT

A method of controlling the impurities in a cyclosporin A eye gel. A high performance liquid chromatography is performed, and chromatographic conditions are as follows: the detection wavelength is 210-230 nm; the column temperature is 60-68° C.; the flow rate is 0.8-1 ml/min; and the mobile phase A is: THF-water-phosphoric acid. The method of controlling impurities solves the problem of excipients interference and separation of many impurities at the same time, it also provides an effective method for the formulation of quality standard of impurities in this kind of preparation.

### CROSS REFERENCE TO THE RELATED APPLICATIONS

This application is based upon and claims priority to Chinese Patent Application No. 201711391728.1, filed on Dec 21, 2017, the entire contents of which are incorporated herein by reference.

### TECHNICAL FIELD

The invention relates to the field of analytical chemistry, in particular to an impurity control method for cyclosporin A eye gel.

### BACKGROUND

Cyclosporine A (CyA) is a cyclic polypeptide consisting of 11 amino acids and is an active metabolite of a fungus in the soil. In 1978, CyA was first applied to clinical kidney transplantation in UK. After that, CyA was used for transplantation of liver, heart, lung, pancreas, bone marrow and other organs. All of them achieved satisfactory results and significantly improved the survival rate of patients. In 1984, cyclosporine A entered Chinese and formed a triple immunosuppressive regimen of “cyclosporine A+azathioprine+hormone”, which greatly improved the survival rate of transplantation and also significantly reduced the incidence of acute rejection after transplantation. As a potent immunosuppressive agent, CyA has been researched more and more in recent years and is widely used in the treatment of various diseases.

Cyclosporine is poorly soluble. In order to making Cyclosporine into a uniform gel or liquid preparation, it is needed to be dissolved. Usually, cosolvents such as polyoxyethylene castor oil, castor oil, Tween and the like are added to ensure dissolution of cyclosporineand no precipitation during storage and use. However, the solubilizer generally has a strong UV peak, and there is a risk of excipient interference, and there are many process impurities and degradation impurities for cyclosporine. Existing impurities control methods for various raw materials cannot effectively detect these impurities.

Cyclosporine gel, cyclosporine ophthalmic emulsion and cyclosporine injection all contain solubilizers. These solubilizers have the following types: polyoxyethylene castor oil 35, polyoxyethylene castor oil 40, castor oil, etc.. These solubilizers have high UV response values, and strong peaks, and similar polarities to cyclosporine, so these excipients often have a significant impact on cyclosporine impurity detection. There is no patent publication on controlling impurities in cyclosporine preparations for the time being. There are no impurity control items and control methods in the literature and the cyclosporine preparations included in the pharmacopoeias of countries.

For ophthalmic preparations, the state requires management according to an injection, so the impurities control of the preparation is very strict. However, at present, in domestic and foreign patent documents and pharmacopoeias, no methods and limits for impurities are proposed for cyclosporine capsules, cyclosporine injections, etc., thus bringing risks to clinical applications and reducing the quality of products. How to solve the problem of excipient interference in the detection and the separation and control of many impurities has become an urgent problem to be solved.

The method that has been published for the control of impurities in cyclosporine is the method for cyclosporine drug substance in Chinese pharmacopoeia. The analytical methods are:

Chromatographic column: C₁₈ chromatographic column (150×4.6 mm, 5 μm)

The mobile phase: acetonitrile-water-methyl t-butyl ether-phosphoric acid (430:520:50:1)

Flow rate: 1.0 ml/min

Column temperature: 70° C.

Wave length: 220 nm

Sample volume: 80 μl

The problems of this method are: the specificity of detecting multiple impurities at the same time is not good, the separation degree of impurity from main peak is poor, the peaks of some impurities come out too early, and blank excipients interfere with detection of impurities having early peaks. The chromatogram of the mixed standard sample is shown in FIG. 1. As can be seen from FIG. 1, the first two impurities are poorly separated, and baseline separation cannot be achieved for the impurities behind the main peak, and controls of multiple known impurities cannot be achieved simultaneously. The separation between impurities, impurity and main peak can not be achieved, the peak interference of excipients is serious, which affects the detection of impurity. Therefore, it is necessary to establish a new method for the detection of related substances, which can detect and isolate the above six known impurities (cyclosporine B, cyclosporin C, cyclosporine D, cyclosporin H, isocyclosporine A, isocyclosporine H) and unknown impurities.

### SUMMARY

An object of the present invention is to provide a method for controlling the impurities of cyclosporin A eye gel, which are determined by high performance liquid chromatography, wherein the chromatographic conditions are as follows:

The detection wavelength is 210-230 nm;

The column temperature is 60-68° C.;

The flow rate is 0.8-1 ml/min;

Mobile phase: THF-water-phosphoric acid.

Preferably, the mobile phase THF-water-phosphoric acid has a volume ratio of 400:600:1.5-2.

Preferably, the column temperature is 65° C.

Preferably, the column is octadecylsilane-bonded silica gel as a filler (300 mm*3.9 mm, 4 um).

Preferably, the detection wavelength is 220 nm.

Preferably, the column is a waters column, a Thermo column, a Pheromone column or a YMC column.

More preferably, the mobile phase has a volume ratio of THF-water-phosphoric acid of 400:600:1.58.

The method of the present invention adopts the chromatographic column octadecylsilane bonded silica gel as a filler (300 mm×3.9 mm, 4 μm); the mobile phase is THF-water-phosphoric acid (400:600:1.58); the detection wavelength is 220 nm; the column temperature is 65° C., the flow rate is 0.8 ml/min. The method can detect and isolate six known impurities cyclosporine B, cyclosporine C, cyclosporine D, cyclosporine H, isocyclosporine A, isocyclosporine H and unknown impurities, as shown in FIG. 2. The limit for single known and unknown impurity is set at 1%. Animal experiments and clinical trials show that this method controls the preparation to achieve the standard preparation, showing good safety.

### BRIEF DESCRIPTION OF THE DRAWINGS

FIG. 1 is an adaptive mixed standard chromatogram of the impurity system in Chinese Pharmacopoeia.

FIG. 2 is an adaptive mixed standard chromatogram of the impurity system of example 12;

FIG. 3 is an adaptive mixed standard chromatogram of the impurity system of example 1;

FIG. 4 is an adaptive mixed standard chromatogram of the impurity system of example 2;

FIG. 5 is an adaptive mixed standard chromatogram of the impurity system of example 3;

FIG. 6 is an adaptive mixed standard chromatogram of the impurity system of example 4;

FIG. 7 is an adaptive mixed standard chromatogram of the impurity system of example 5;

FIG. 8 is an adaptive mixed standard chromatogram of the impurity system of example 6;

FIG. 9 is an adaptive mixed standard chromatogram of the impurity system of example 7;

FIG. 10 is an adaptive mixed standard chromatogram of an impurity system in example 8;

FIG. 11 is an adaptive mixed standard chromatogram of the impurity system of example 9;

FIG. 12 is an adaptive mixed standard chromatogram of the impurity system in example 10; and

FIG. 13 is an adaptive mixed standard chromatogram of the impurity system in example 11.

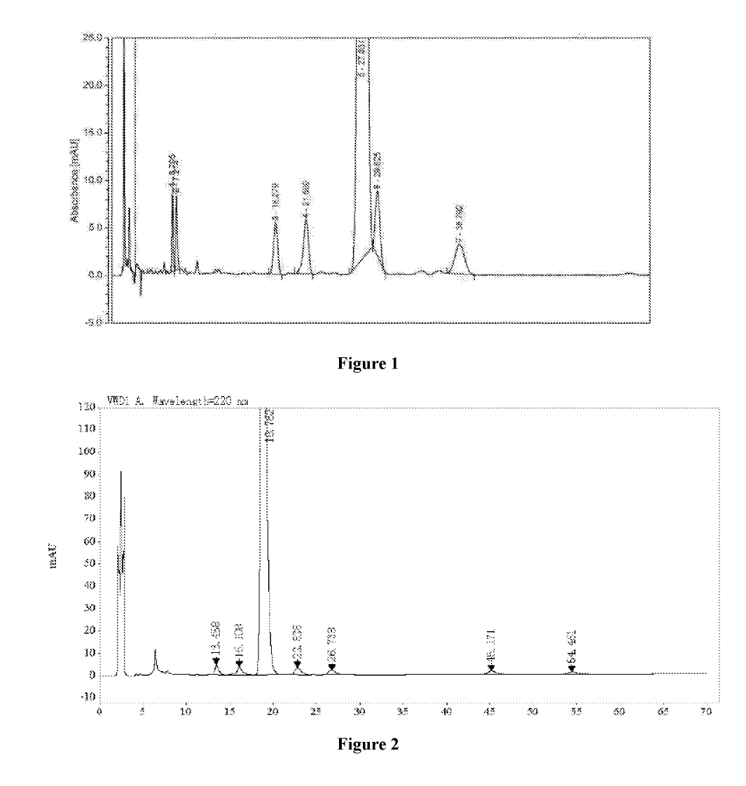

### DETAILED DESCRIPTION

The present invention discloses a method for controlling impurities of cyclosporin A eye gel, and those skilled in the art can learn from the contents of the disclosure and appropriately improve the process parameters. It is to be understood that all such alternatives and modifications are apparent to those skilled in the art and are considered to be included in the present invention. The method of the present invention has been described in terms of preferred embodiments, and it is apparent that the method and application described herein may be changed or modified and combined to implement and apply the present invention without departing from the content, spirit, and scope of the invention.

In order to make those skilled in the art better understand the technical solutions of the present invention, the present invention will be further described in detail below with reference to specific examples.

### EXAMPLE 1

### Chromatographic Conditions

Mobile phase: Acetonitrile-water-Methyl-t-butyl Ether-phosphoric acid (430:520:50:1)
Wavelength: 210 nm;
Flow rate: 1.0 ml/min;
Column temperature: 70° C.;
Sample volume: 80 ul;
Chromatographic column: watersC18 (150mm*4.6mm, 5 um) filler

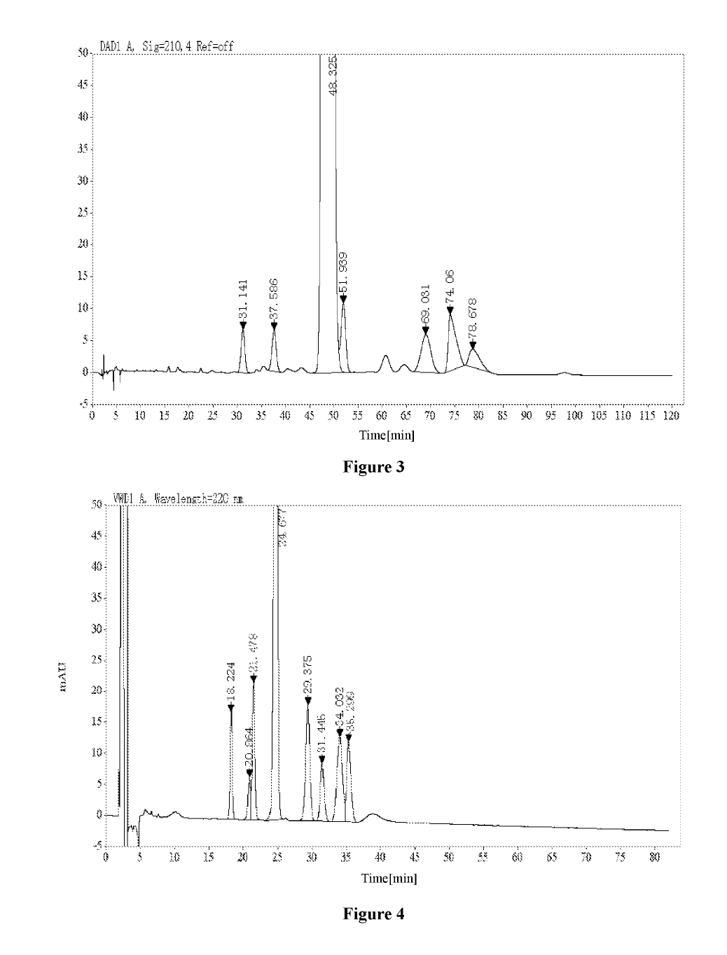

The solution chromatogram for the systematic application is shown in FIG. 3. The results showed that there are many interference peaks interfering with the effective detection of impurities, and the separation degree of the main peak from the latter impurity (retention time 51.9 min) is poor, the last two impurities do not achieve baseline separation, therefore this method can not be used to detect the related substances of cyclosporine A eye gel.

### EXAMPLE 2

### Chromatographic Conditions

Reference is made to article “Assay of Cyclosporin and its degradation products in cyclosporine capsules by HPLC method” of the Chinese Journal of antibiotics, Vol. 27, No. 4, April, 2002. The flow rate is 1 ml/min, HPLC cloume is HypersilBDSC18250 mm*4.6 mm,5 um, Mobile phase: water-THF-0.4 mol/L N-propylamine phosphate solution (0.4 mol/L N-propylamine solution, adjusting pH value to 2.6 with phosphoric acid)=590: 400: 10, column temperature is 70° C., and the wavelength is 220 nm.

The chromatogram is shown in FIG. 4. The results showed that the separation degree of impurity was poor when the retention time was 34.0 min or 35.3 min, baseline separation was not achieved.

### EXAMPLE 3

### Chromatographic Conditions

On the basis of example 2, the proportion of organic phases was reduced. The specific conditions are as follows: the flow rate is 1 ml/min, the HPLC column is Hypersil BDS C18 250 mm*4.6 mm, 5 u, Mobile phase: [Water-0.4 mol/L N-propylamine phosphate solution(0.4 mol/L N-propylamine solution, adjusting pH value to 2.6 with phosphoric acid)=(885:15)]−THF=650:350, column temperature is 70° C., and the wavelength is 220 nm.

The chromatogram is shown in FIG. 5. The results show that when the retention time was 69.4 min and 71.0 min, baseline separation of impurities was not achieved.

### EXAMPLE 4

### Chromatographic Conditions

On the basis of example 3, the chromatographic column was changed. The specific conditions are as follows: the flow rate is lml/min, the HPLC column is Waters Nova-Pak C18 350 mm*3.9 mm, 4 um, the mobile phase: [Water-0.4 mol/L N-propylamine phosphate solution(0.4 mol/L N-propylamine solution, adjusting pH value to 2.6 with phosphoric acid)=(885:15)]−THF=650: 350, the column temperature is 70° C., and the wavelength is 220 nm.

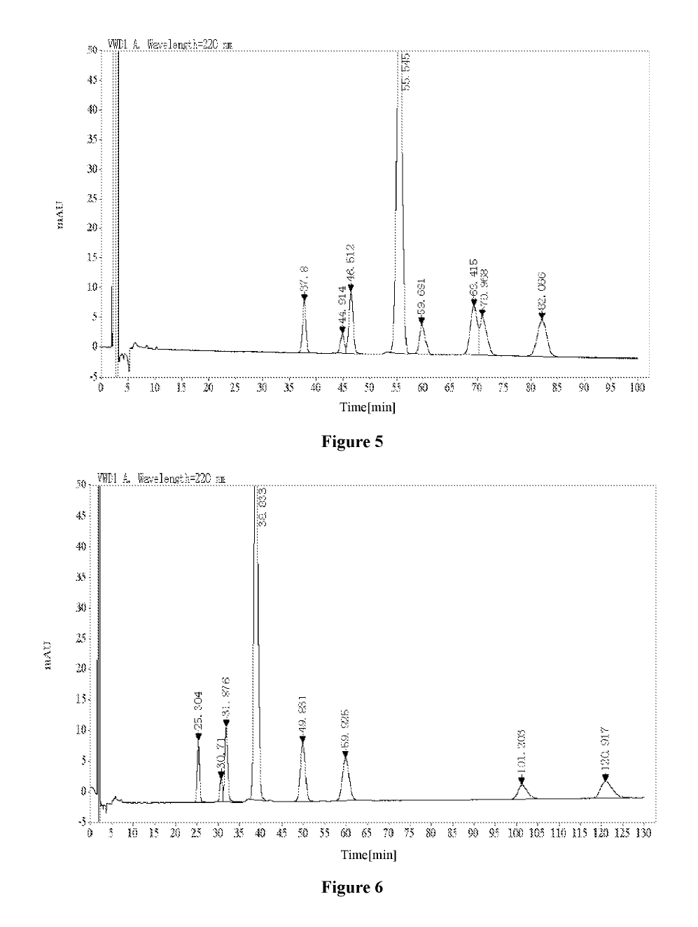

The chromatogram is shown in FIG. 6. The result showed that the collecting time was too long , which was 130 minutes.

### EXAMPLE 5

### Chromatographic Conditions

On the basis of example 4, the isometric condition is changed to a gradient condition. Considering that the baseline noise of the mixed mobile phase through the instrument proportional valve is high, a mixed mobile phase was prepare. The specific conditions are as follows: the flow rate is lml/min, the HPLC column is Waters Nova-Pak C18 350 mm*3.9 mm, 4 um, the mobile phase A: [Water-0.4 mol/L N-propylamine phosphate solution(0.4 mol/LN propylamine solution, adjusting pH value to 2.6 with phosphoric acid)=(885: 15)]−THF=650:370, the mobile phase B: [Water-0.4 mol/L N-propylamine phosphate solution (0.4 mol/LN propylamine solution, adjusting pH value to 2.6 with phosphoric acid)=(885: 15)]−THF=600:400, the column temperature is 70° C., and the wavelength is 220 nm.

### Gradient Srocedure:

| Time (min) | A % | B % |
|---|---|---|
| 0 | 100 | 0 |
| 40 | 100 | 0 |
| 50 | 0 | 100 |
| 80 | 0 | 100 |
| 81 | 100 | 0 |
| 90 | 100 | 0, |

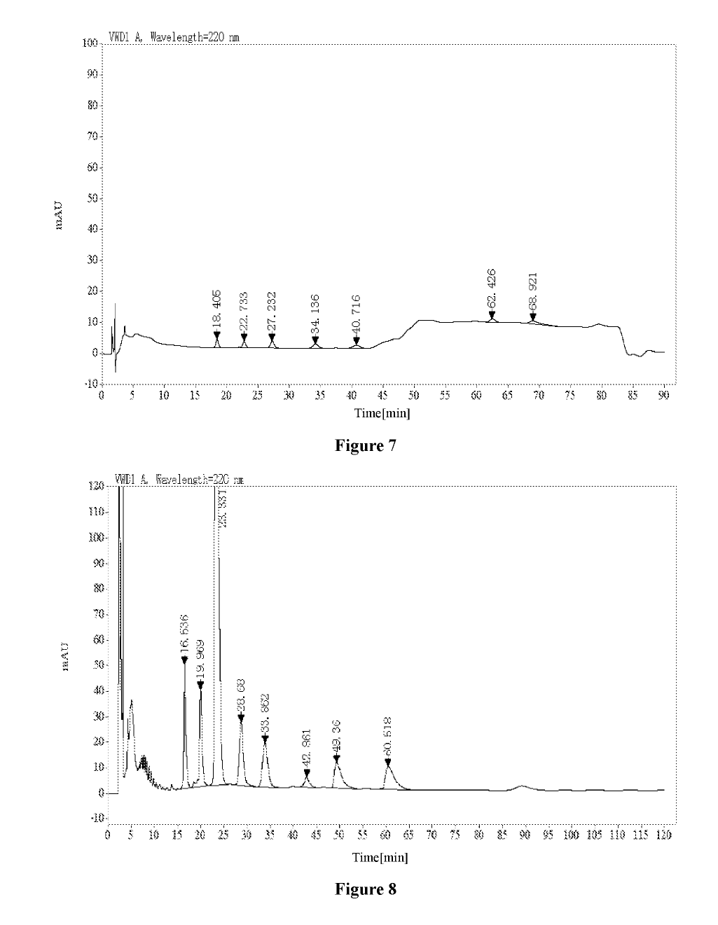

The chromatogram is shown in FIG. 7. The results show that the baseline noise is large and impurity detection is interfered.

### EXAMPLE 6

chromatographic conditions: It plans to remove N-propylamine from the mobile phase. The specific chromatographic conditions are as follows:

The flow rate is 0.7 ml/min, the HPLC column is Waters Nova-Pak C18 350 mm*3.9 mm, 4 um, the mobile phase: THF-water-85% phosphoric acid=400:600:1.58 ml, the column temperature is 70° C., and the wavelength is 220 nm.

The chromatogram is shown in FIG. 8. The results showed that the acquisition time was 120 min and the acquisition time was too long.

### EXAMPLE 7

### Chromatographic Conditions

It plans to change the amount of phosphoric acid in the mobile phase. The specific chromatographic conditions are as follows: the flow rate is 0.8 ml/min, the HPLC column is Waters Nova-Pak C18 350 mm*3.9 mm, 4 um, the mobile phase: THF-water-85% phosphoric acid=400:600:1.8, the column temperature is 65° C., and the wavelength is 220 nm.

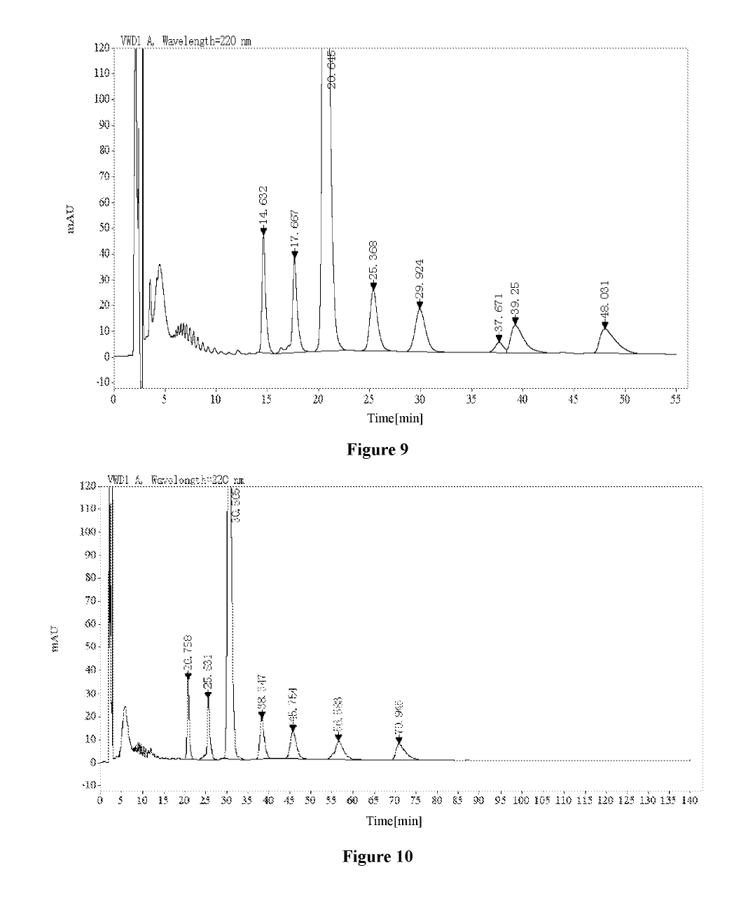

The chromatogram is shown in FIG. 9. The results show that the detection of impurity at 39.3 min was interfered by excipient peak at 37.7 min.

### EXAMPLE 8

### Chromatographic Conditions

It plan to reduce the proportion of organic phase in the mobile phase. The specific chromatographic conditions are as follows: the flow rate is 0.8 ml/min, the HPLC column is Waters Nova-Pak C18 350 mm*3.9 mm, 4 um, the mobile phase: THF-water-85% phosphoric acid=380:620:1.58, the column temperature is 65° C., and the wavelength is 220 nm.

The chromatogram is shown in FIG. 10. The results showed that the peak of excipient at 140 min was not finished and the collecting time was too long.

### EXAMPLE 9

### Chromatographic Conditions

Mobile phase: Water-tetrahydrofuran-85% phosphoric acid (600:400:1.58)
Wavelength: 220 nm;
Flow rate: 0.7 ml/min;
Column temperature: 70° C.;
Sample volume: 80 ul;
Chromatographic column: C18, 300 mm*3.9 mm, 4 um

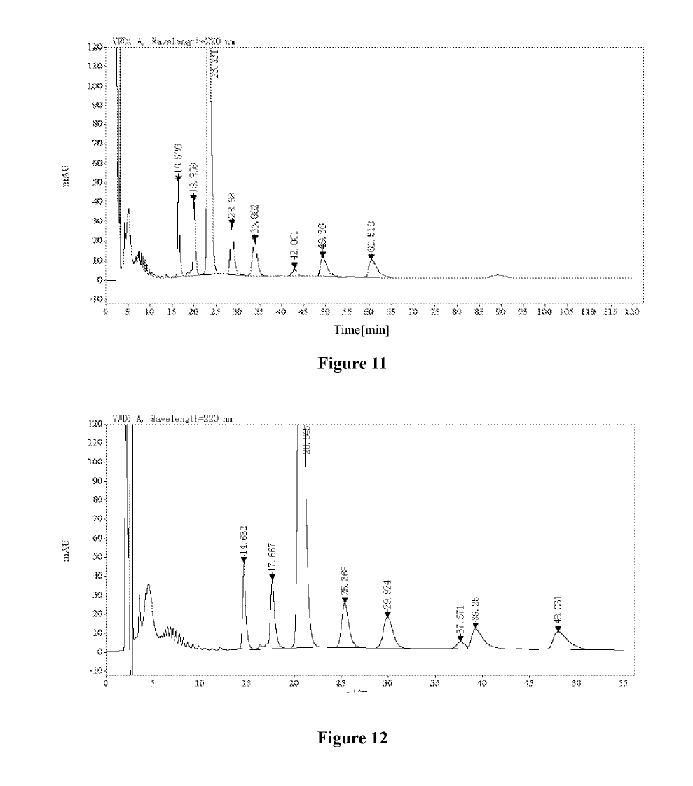

The chromatogram of the solution for systematic application is shown in FIG. 11. What to be improved is: there is excipient peak at 90min and collecting time 120 minutes is too long.

### EXAMPLE 10

### Chromatographic Conditions

Mobile phase: Water-tetrahydrofuran-85% phosphoric acid (600:400:1.80);
Wavelength: 220 nm;
Flow rate: 0.8 ml/min;
Column temperature: 65° C.;
Chromatographic column: C18, 300 mm*3.9 mm, 4 um
Sample volume: 100 ul;

The chromatogram of the solution for systematic application is shown in FIG. 12.

What to be improved is: the excipient peak 37 is not completely separated from the latter impurity and interferes with the impurity detection.

### EXAMPLE 11

### Chromatographic Conditions

Mobile phase: Water-tetrahydrofuran-85% phosphoric acid (600:400:1.58);
Wavelength: 220 nm;
Flow rate: 0.8 ml/min;
Column temperature: 65° C.;
hromatographic column: C18, 300 mm*3.9 mm, 4 um
Sample volume: 100 ul;

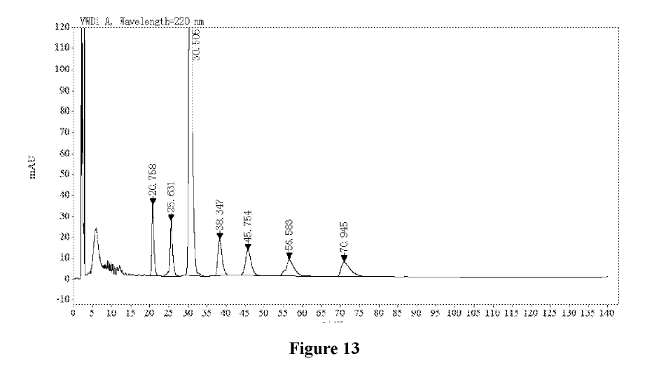

The chromatogram of the solution for systematic application is shown in FIG. 13.

What to be improved is: the peak retention time of excipient was 140 minutes and the collecting time was too long.

### EXAMPLE 12

### Chromatographic Conditions

Mobile phase: Water-tetrahydrofuran-85% phosphoric acid (600:400:1.58);
Wavelength: 220 nm;
Flow rate: 0.8 ml/min;
Column temperature: 65° C.;
Chromatographic column: C18, 300 mm*3.9 mm, 4 um
Sample volume: 100 ul;

The chromatogram of the solution for systematic application is shown in FIG. 2.

The above description only shows preferred embodiment of the present invention, and it should be noted that those skilled in the art can also make a number of improvements and modifications without departing from the principles of the present invention, and these improvements and modifications should be considered falling within the scope of protection of the present invention.

1. A method of controlling the impurities in a cyclosporin A eye gel, comprising:
performing high performance liquid chromatography, wherein, chromatographic conditions are as follows:
a detection wavelength is 210-230 nm;
a column temperature is 60-68° C.;
a flow rate is 0.8-1 ml/min; and
a mobile phase is THF-water-phosphoric acid.

2. The method of controlling impurities according to claim 1, wherein a volume ratio of THF, water, and phosphoric acid in the mobile phase is 400:600:1.5-1.8.

3. The method of controlling impurities according to claim 1, wherein the column temperature is 65° C.

4. The method of controlling impurities according to claim 1, wherein the chromatographic column has octadecylsilane-bonded silica gel as a filler.

5. The method of controlling impurities according to claim 1, wherein the detection wavelength is 220 nm.

6. The method of controlling impurities according to claim 1, wherein the column is a waters column, a Thermo column, a Pheromone column or a YMC column.

7. The method of controlling impurities according to claim 1, wherein the volume ratio of THF, water, and phosphoric acid in the mobile phase is 400:600:1.58.
